(19) United States
(12) Patent Application Publication
(10) Pub. No.: US 2022/0364131 A1
(43) Pub. Date: Nov. 17, 2022

(54) PROCESS FOR MANUFACTURING ORGANIC CHEMICALS AND/OR DISTILLATE HYDROCARBON FUELS FROM WASTE TEXTILES

(71) Applicant: SHARETEX AB, Bjärred (SE)

(72) Inventor: Lars STIGSSON, BJÄRRED (SE)

(21) Appl. No.: 17/765,643

(22) PCT Filed: Aug. 6, 2020
(86) PCT No.: PCT/SE2020/050768

§ 371 Date: Mar. 31, 2022

(30) Foreign Application Priority Data
Oct. 4, 2019 (SE) 1951132-8
Dec. 11, 2019 (SE) 1951429-8
Dec. 16, 2019 (SE) 1951465-2

(51) Int. Cl. C12P 19/02; C12P 7/18; C12P 17/10; C12P 7/10; C10G 1/10; C10G 5/06; C07C 29/136; C07C 29/80; C12P 7/46; C10G 3/00; C12P 7/56

(52) U.S. Cl. CPC C12P 19/02; C12P 7/18; C12P 17/10; C12P 7/10; C10G 1/10; C10G 5/06; C07C 29/136; C07C 29/80; C12P 7/46; C10G 3/42; C12P 7/56; C10G 2300/1003; C10G 2400/08

(57) ABSTRACT

The present disclosure relates to a process for manufacturing organic chemicals and/or distillate hydrocarbon fuels from waste textiles comprising cellulosic fibers, wherein the process includes providing waste textiles comprising cellulosic fibers, processing the waste textiles into an aqueous slurry of comminuted waste textiles, saccharification of the comminuted waste textiles into monomer sugars in the presence of a catalyst; and processing the monomer sugars into organic chemicals and/or distillate hydrocarbon fuels.

### TECHNICAL FIELD

The present disclosure relates to a process for manufacturing organic chemicals and/or distillate hydrocarbon fuels from cellulosic waste textiles.

### TECHNICAL BACKGROUND

Around 90 million tons of textile fibers were produced in 2014 and the market is expected to grow steadily over the next few years, exceeding well over 120 million tons in 2025. This enlargement of the textile sector poses an environmental challenge generating large amounts of waste. It is estimated that less than 10% of all used textile products are recycled today. Currently, landfilling and incineration are the most common techniques for managing this waste.

In line with the principles of circular economy, it will be desirable to develop new valorization strategies that can recover and recycle textile fibers so that this resource can be reintroduced to the market and consumers at a higher value than that of incineration (or landfill).

There are processes available for fiber recovery from waste textile fibers, which can be favorable from a circular perspective (such as re-use in the second-hand market) and for regeneration of new textiles where the fibers remain in the material system. However, the polymers building up the textile tend to be depolymerized during use and washing, and not all recycled material is suitable for recycling. Moreover, certain manufactured cellulosic fiber materials, such as viscose, have polymers with an intrinsic low molecular weight, which is further lowered during use.

Therefore, there is a need for new technologies and climate effective processes wherein waste cellulosic textiles, independent of wear and tear, can at least partly be used as a feedstock for more valuable products.

### SUMMARY AND OBJECTIVES

It is an object of at least some of the examples of the present disclosure to provide an improvement over the above described techniques and known art in textile recycling.

A further object of at least some examples of the present disclosure is to provide a process to recover value from worn cellulosic textiles and textile waste, specifically such textiles having depolymerized cellulose chains. For the avoidance of doubt, the waste textiles may comprise recycled textiles.

A further object of at least some examples of the present disclosure is to provide recovery and upgrade of waste textiles to jet fuels and/or valuable organic chemicals.

At least some of these and other objects and advantages that will be apparent from the description is achieved by a process for manufacturing organic chemicals and/or distillate hydrocarbon fuels from waste textiles comprising cellulosic fibers according to a first aspect of the disclosure. The process comprises:
providing waste textiles comprising cellulosic fibers;
processing the waste textiles into an aqueous slurry of comminuted waste textiles;
saccharification of the comminuted waste textiles into monomer sugars in the presence of a catalyst; and
processing the monomer sugars into organic chemicals and/or distillate hydrocarbon fuels.

Organic chemicals are a broad class of substances containing carbon and its derivatives such as alcohols, ketones, aldehydes and lactams. Organic chemicals can be manufactured for example by synthesis from fossil oil derivatives or from biomass by biotechnological processes. The organic chemicals may be fine organic chemicals. The organic chemicals may be so called bio-organic chemicals with an origin from biomass.

Comminuted waste textiles are understood to mean particulates that are preferably 10×10 mm or less as an example area but may be larger.

Waste textiles are understood to mean used textiles such as used clothing, home textiles, recycled textiles and recycled textile fibers, waste textiles from textile production, etc. Polymer chains in the waste textiles may be depolymerized for example by washing, wear and tear, as for example in the case of at least some cellulosic recycled textile fiber. Cellulose polymer chains may be shortened also by the manufacturing process of textile fibers. One such example is viscose. Degree of depolymerization may be described by the intrinsic viscosity of the polymers.

The distillate hydrocarbon fuels may be in the jet fuel range, such as having a carbon number of the hydrocarbons in the range of C8-C16, and/or having a density of 775.0-840.0 g/l, and/or a freezing point of −40-−50° C., and/or a boiling point of 170-180° C.

The distillate hydrocarbon fuels may be in other fuel ranges, such as in a range suitable for gasoline.

The waste textiles are advantageously provided in form of an aqueous slurry of comminuted waste textiles.

The saccharification of comminuted cellulosic waste textiles into monomer sugars is performed by saccharification of the aqueous slurry of comminuted waste textiles.

The monomer sugars comprise glucose.

A glucose yield may be higher than about 90%, preferably higher than about 95%, calculated on sugar content of raw waste textiles material.

Saccharification of the comminuted waste textiles may be performed by hydrolysis catalyzed by an acidic catalyst.

The acid catalyst in the acid hydrolysis may be sulfuric acid or a solid acid catalyst.

At least a portion of the acid catalyst may be separated from the formed glucose and is optionally restored and thereafter recycled to the saccharification step.

Saccharification of the comminuted waste textiles may be performed by treatment with saccharification enzymes.

Processing the monomer sugars to organic chemicals and/or distillate hydrocarbon fuels may be performed by fermentation.

Fermentation of the monomer sugars may comprise fermentation of the monomer sugars to an organic alcohol, organic acid or to a lactam.

The process may be directed to the manufacturing of distillate hydrocarbon fuels and processing the monomer sugars to an alcohol as an intermediate step may be performed by fermentation.

The process may be directed to the manufacturing of distillate hydrocarbon fuels, and may further comprise:
separating alcohol from a fermentation broth,
concentrating the alcohol by distillation; and
further treating the concentrated alcohol in one or more steps to form distillate hydrocarbon fuels in a carbon number range of C8 to C16.

The step of further treating the concentrated alcohol may comprise at least one of dehydration, oligomerization, and hydrogenation.

The step of further treating the concentrated alcohol may be performed in a petroleum refinery.

The alcohol may be ethanol.

The alcohol may be butanol or isobutanol.

Isobutene may be produced directly from the ethanol as an intermediate olefin prior to oligomerization.

Processing of the monomer sugars to organic chemicals and/or distillate hydrocarbon fuels may be performed by fermentation and/or a catalytic conversion process.

The process may be directed to the manufacturing of organic chemicals, and processing of the monomer sugars to organic chemicals may be performed by fermentation as an intermediate step.

The fermentation of monomer sugars may comprise conversion of monomer sugars to 1,4 butanediol or caprolactam and/or catalytic conversion of monomer sugars to 2,5 furan dicarboxylic acid.

The catalytic conversion and/or fermentation of monomer sugars may comprise conversion of sugars to succinic acid, lactic acid or malonic acid

Processing the waste textiles into a slurry of comminuted waste textiles is performed by disintegration of the waste textiles by a chemical, thermochemical, or mechanical treatment.

Disintegration of the waste textiles by a chemical treatment may include treatment by sodium carbonate and/or sodium hydroxide or an acid such as sulfuric acid.

Disintegration of the waste textiles by a thermochemical treatment may include steam explosion process and/or hydrothermal treatment.

Disintegration of the waste textiles by a mechanical treatment may include at least one of a grinding, milling, and/or chopping.

The process may further comprise pre-processing the waste textiles prior to disintegrating the waste textiles.

Pre-processing of the waste textiles may comprise mechanical and/or chemical separation of polyester, cotton fabric or fibers from the waste textiles

The pre-processing of the waste textiles may comprise mechanical sorting by fiber composition using by NIR/VIS technology (near infrared, visible ray) for fiber detection.

The pre-processing of the waste textiles may comprise a steam explosion process.

The pre-processing of the waste textiles may comprise a hydrothermal treatment.

The process may advantageously be integrated in a kraft, sulfite or organosolv pulp mill.

The waste textiles may comprise cotton (preferably low-quality cotton), viscose, and/or lyocell cellulosic fibers.

The waste textiles may comprise cold alkali fibers such as carbamate fibers.

The pre-processing of the waste textiles may comprise pre-treating of the waste textiles off-site.

The waste textiles may further comprise synthetic fibers, and wherein during the fermentation or catalytic conversion, the synthetic fibers form an inert sludge, the inert sludge being separated from the monomer sugars, formed in the fermentation step.

The inert sludge may further be treated by a chemical or thermal process to recover an energy or material value of the synthetic fibers.

Synthetic fibers are understood to mean non-cellulosic fibers.

The synthetic fibers may comprise polyester. Furthermore, the synthetic fibers may comprise at least one of polyamide nylon, PET or PBT polyester, phenol-formaldehyde (PF), polyvinyl chloride fiber (PVC), polyolefins (PP and PE) olefin fiber, acrylic polyesters, aromatic polyamides, polyethylene, elastomers and polyurethane fibers.

The process may further comprise pyrolyzing the inert sludge to form a synthesis gas and condensing the gas to form a hydrocarbon liquid.

The hydrocarbon liquid may be transported to a petroleum refinery for hydro-processing into distillate fuels.

The inert sludge may be used as a feedstock for preparation of new synthetic fibers.

The cellulose polymers that are building blocks in cellulosic textiles and fabrics such as viscose and cotton waste textiles charged to the process of the disclosure may have large fraction, preferably over 50% by weight of polymers with an average intrinsic viscosity lower than an intrinsic viscosity IV of 600 as determined by ISO5351:2010.

In line with the above, according to one alternative of the method according to the present invention, the waste textiles charged to the process comprise a large fraction, preferably over 50% by weight of waste textiles, of cotton, viscose or cold alkali fibers having an average cellulosic polymer molecular chain length lower than corresponding to an intrinsic viscosity (IV) of 600.

The acid hydrolysis may be performed in two steps with different acid concentrations in each step.

At least a portion of the acid hydrolysis may be performed in the presence of a solid acid catalyst.

At least a portion of the of the acid catalyst may be recycled to the saccharification step.

### BRIEF DESCRIPTION OF THE DRAWINGS

The present disclosure will, by way of example, be described in more detail with reference to the appended schematic drawings, which show examples of the present disclosure.

FIG. 1 shows a process for manufacturing organic chemicals and/or distillate hydrocarbon fuels from waste textiles comprising cellulosic fibers.

FIG. 2 shows a schematic block-diagram of a two-step hydrolysis process with weight contents of each step of the process.

### DETAILED DESCRIPTION

The valorization of waste cotton and viscose fibers holds great opportunities as it can ease the environmental burden of the waste textile management and simultaneously help to develop a functioning bio-economy.

The reason for this is that waste cotton and viscose fibers may be an inexpensive source of cellulose for ethanol production or for production of other organic chemicals. Feedstock cost is one of the main contributions to the production cost for biochemicals such as bioethanol, corresponding to about 40% or more of the production cost.

The present disclosure is also directed to an innovative route for upgrade and valorization of textile waste mixtures comprising synthetic fibers such as polyester, cellulosic fibers such as cotton and blends of various fibers. Polyester is by far the largest textile fiber today, but it is expected that fibers made of fossil feedstocks may decline in the future. Associated problems with micro-plastic in the oceans contribute to the disadvantages of polyester.

One example of the present disclosure is based on using sorted textile fiber waste comprising substantially of at least one of cotton, viscose and/or lyocell, and cold alkali fibers such as conventional cold alkali fiber or carbamate fiber. Cold alkali fibers are described in Cellulose in “NaOH-water based solvents: a review” Tatiana Budtova, Patrick Navard. Cellulose in NaOH-water based solvents: a review. Cellulose, Springer Verlag, 2016, 23 (1), pp. 5-55. 10.1007/s10570-015-0779-8. hal-01247093.

In particular, cellulosic fiber waste where the average intrinsic viscosity of the cellulose polymers is lower than about 600, as determined by using ISO5351:2010, and therefore not being suitable for manufacturing of new regenerated textile fibers, can advantageously be converted to valuable organic chemicals in accordance with the present disclosure.

Alternative processes have previously been explored for conversion of cellulosic materials into monomer sugars such as glucose. These processes have mainly been developed using lignocellulosic biomass, e.g. from plants, which also comprise both lignin and hemicellulose, as a feedstock. The structure of lignocellulose is complex and to a certain extent resistant to chemicals and hydrolysis. Hydrolysis of lignocellulosic biomass may also result in unwanted byproducts which can inhibit subsequent treatment such as fermentation. Thus, it is important to remove the lignin and hemicellulose from lignocellulosic material through pretreatment.

Using feedstock such as cotton or viscose therefore reduces the operating time and operational costs associated with the removal of lignin and hemicellulose. Cotton and viscose waste textiles as a feedstock are both sustainably beneficial as well as economically beneficial due to the large availability of the resource and the low price of it. The increasing global population has an increasing demand of textile per capita which has subsequently led to the global textile production expanding at a very high rate with nine times the textile production now in 2020 compared to 1980.

For ideal waste management, the textiles should be reused rather than recycled and recycled rather than discarded. However, textile fibers become damaged over time. After having been recycled, the fibers become shorter and the degree of polymerization decreases, hindering the possibility of mechanically or chemically creating new fibers and fabric from the material. It is mainly this material which is intended for conversion to glucose in accordance with the present disclosure. These used textiles, low quality and degraded cotton or other cellulosic fibers such as viscose and cold alkali fibers, are characterized by having cellulose polymers with an intrinsic viscosity (IV) of less than about 600. The IV describes the chain length and weight properties of the fibers and may be used to calculate the degree of polymerization (DP) of the material. It should be noted that the molecular weight of the cellulose polymers in a cellulosic substrate (such as for instance dissolving pulp or viscose fabric) may be determined by using intrinsic viscosity (IV). The IV may be determined by using a standard method, such as ISO5351:2010. When a value of the intrinsic viscosity is set, this may be used to calculate a value of the degree of polymerization (DP), for instance via DP=0.7277*(IV)\hat{ }1.105. For instance, a DP value in the range of 185-325 then corresponds to a value of the intrinsic viscosity (IV) of about 150-250 mL/g.

In the following description, glucose is used as an example of a monomer sugar.

One example of the present disclosure is based on using a blend of synthetic fibers such as polyester and cellulosic fibers including cotton, viscose and/or lyocell and cold alkali fibers. Such fiber blends are processed into an alcohol such as ethanol or into organic chemicals/intermediate chemicals in accordance with the processes disclosed herein, i.e. steam explosion treatment/fractionation, alkaline hydrolysis, enzymatic saccharification or acid hydrolysis of the cellulose polymers to sugars, followed by fermentation, and separation of the products from fermentation. The polyester fraction is merely present as an inert sludge through these process steps and is recovered as a by-product sludge. The inert sludge comprising at least partly decomposed polyester material is either recycled to become a feedstock for new polyester by known methods or directly or indirectly injected into a chemical recovery boiler of a pulp mill or and on or offsite pyrolysis unit wherein the material is gasified under oxygen deficiency. The formed pyrolysis gases are condensed and separated into a hydrocarbon liquid. This liquid can be distilled, and the distillate can either be used as a fuel directly or be exported to a petroleum refinery for hydro-processing and upgrading to distillate fuels such as jet fuels.

In one example, the cellulose in waste textiles is used to produce ethanol by saccharification and fermentation. Thereafter the ethanol is dehydrated to form a dry and water-free alcohol. The dry alcohol is subsequently further upgraded by removing water from the ethanol molecule, oligomerization, and hydrogenation to form drop-in jet fuel molecules. Alternatively, ethanol can be dehydrated to ethylene, and the ethylene can be used for production of polyethylene.

In an alternative example, the glucose rich sugar stream recovered from saccharification is used for manufacturing of organic chemicals and intermediates such as 1,4 butanediol, caprolactam and/or FDCA (furan-dicarboxylic acid) by catalytic, biocatalytic and/or microbial processes.

While the process and unit operations of the disclosure disclosed herein can be operated stand-alone, it can be advantageous to integrate the process with kraft, sulfite or organosolv pulp mills, as any energy surplus from the mill operations may be used to power the process, but mainly for providing combined recycling and recovery of process chemicals in pulp process machinery. Green liquor, a process stream in the kraft pulp mill recovery cycle, can be used as a pretreatment agent to make waste cotton or viscose fibers more amenable to enzymatic saccharification and subsequent ethanol and/or organic chemicals production. One of the main components of green liquor is sodium carbonate, which has previously been proven to be a suitable pre-treatment agent applied to waste cellulose such as cotton waste prior to enzymatic hydrolysis.

In one example, acid hydrolysis is employed for saccharification of the cellulosic fraction of the waste textile material. Sulfuric acid and/or solid acids are preferably used as catalyst in such acid hydrolysis step.

A major portion of the spent acid or solid acid catalyst is, after optional restoration, recycled after hydrolysis and/or downstream conversion to ethanol and/or organic chemicals. If the process is integrated with a kraft pulp mill, spent acid from tall soap acidulation can be used as acid source.

### Acid Recovery/Catalyst Recycle

For homogeneous acid hydrolysis of the cellulosic fraction in textiles, sulfuric acid is selected as catalyst for both cost and performance reasons. However other acids can be used such as hydrochloric or phosphorous acid. For a process such as acid hydrolysis to be economically viable, the acid should at least partially be recirculated in the system. However, acid recovery can be a high energy-demanding process and, as sulfuric acid cannot be retrieved from distillation as it is not volatile, other methods must be employed. Such methods may be dialysis or electrodialysis by anionic membrane or chromatography methods such as ion exchange chromatography, ion exclusion chromatography or ion retardation resin chromatography.

An alternative to the use of homogeneous acids is to partially or fully use a heterogeneous catalyst such as a solid acid for acidulation. Solid acids have certain advantages in the practice of the present disclosure which is further discussed below.

An alternative to acid recovery is neutralizing the acid with lime, producing gypsum which is discarded. However, although this method is industrial practice, the acid recovery recycling scheme is preferred in accordance with the present disclosure.

A short description of the various acid recycling techniques that can be used in conjunction with saccharification of textile material in accordance with the present disclosure is given below.

### Dialysis by Anionic Membrane

The acidic solution that is to be removed from the fermentation broth contains both anions and cations; sulphate ions and hydrogen ions. Using solely an anion-exchange membrane is efficient for dialysis, as the hydrogen ions are small enough to pass through and will do so as to avoid a negative charge build up on the receiving side of the membrane. Disaccharides have a permeability of less than 1% of that of acids, and thus in effect, only the sulfuric acid is transported through the membrane and separated for reuse. However, for a slurry containing monosaccharides two membranes in series can be applied for filtration. With the streams of each side of the membrane flowing in opposite directions, having the receiving liquid flowing from top to bottom is preferable. This is because the density of this stream increases with an increase of sulfuric acid content. Therefore, this allows avoiding having the heavier liquid mixed back to the lighter liquid. With this construction, the flow of the sulphuric acid is about the same rate as that of the incoming mixture and the concentration of the sulphuric acid stream is close to that of the incoming slurry. This design allows for quite high concentrations of the slurry. If the sulphuric acid stream needs to have a higher concentration concentrated sulphuric acid or SO₃ can be added.

Dialysis can be done either as diffusion dialysis or electrodialysis, with the method of electrodialysis being the more economical alternative. This is due to diffusion dialysis requiring greater membrane costs which outweigh the additional power costs of electrodialysis and due to the acid flux in diffusion dialysis only constituting around 5% of the acid flux of electrodialysis at optimal current density.

As discussed herein it is advantageous to practice a two step procedure for the acid hydrolysis in accordance with the present disclosure. This procedure involves dilution of the acid in the second step. For diffusion dialysis, extensive dilution is a necessity. For electrodialysis, dilution of the hydrolysate solution is beneficial as it increases the current efficiency. A re-concentration step is therefore necessary for both alternatives, as the increased current-efficiency outweighs the re-concentration costs for the electrodialysis.

### Centrifuge Separation

Centrifugal separation of the monosaccharides from the acidic hydrolysate may be employed. Too high temperatures, or inclusion of viscose fibres in the feedstock, may however recycle glucose monomers back into the loop which may degenerate them into degeneration products such as furfural or levulinic acid. Centrifugal separation may be used for separation of sugars in systems practicing enzymatic saccharification of cellulose with acidic pre-treatment.

Specific advantages of centrifugation in comparison to other separation devices are; a continuous separation is possible, the retention time in the device is short (may be seconds), some separation efficiency adjustments are possible on stream without having to stop the process, there is no need for additives and the floor space required is smaller than for other separation processes.

### Ion Exclusion Chromatography (Cationic Resin)

Ion exclusion with a strongly acidic cation exchange resin separates ionic- from non-ionic compounds as the acid is initially eluded due to ion repulsion and as the water and non-ionic fraction of the stream are sorbed to the solid phase for later elution. This differs from ion exchange chromatography as ions do exchange with the resin during ion exchange chromatography, entailing a need for regeneration of the resin, a need which is eliminated with ion exclusion chromatography.

The same resin is used in both ion exchange and ion exclusion chromatography. However, the ionic functionality differs between the two as, for ion exclusion, the ionic functionality is the same as that of the electrolyte, which results in there being no exchange of ions. Several types of resins can be used in the practice of the recycling of sulphuric acid from the saccharification step of the present disclosure such as sulfonated polystyrenes with divinylbenzene cross-linking, where the cross-linking impacts the level of sorption. Due to sulfonic acid functionality, the resin swells in aqueous media and sorbs water and non-ionic solutes. An acidic solution introduced to the resin results in shrinkage, which effects the concentration of the acid/sugar mixture above the resin, which in turn is a cause for dispersion; Dispersion is the arising dilution which results in an unfavourable overlapping of the acidic- and the sugar stream. It is therefore necessary to minimise or compensate for the shrinkage for effective and complete separation of the acids and sugars in the mixture.

Ion exclusion has previously not been considered for industry-scale usage due to scaling considerations, the necessity of small feed volumes, low flux rates and weak electrolyte concentrations to avoid dispersion in order to retain a good separation of the feedstock. However, with improved resin bed performance an efficient operation can be obtained with significantly higher flux rates, feed volumes and electrolyte concentrations than the earlier designs.

### Acid Retardation Resin Chromatography (Anionic Resin)

One of the more conventional methods for acid recovery is that of ion exchange chromatography. However, this method requires the use of large resin beds and therefore hour-long process times, which in turn can lead to a fast degradation of the resin due to the long exposure to chemicals. Recently acid retardation resins comprising a particulate quaternary ammonium resin has been commercialised that does not shrink and expand to the same extent as other resins. The new resin has been successfully applied to acid/sugar streams after sulphuric acid hydrolysis. The non-ionic organic compounds were rejected by the resin and the retained acid was later eluted with water.

Fast flows can be applied, much shorter cycle times and a shorter resin bed for fine particles as well as frequent wash steps of the resin is improving operability. A 98.5 wt % recovery of sulphuric acid as well as a 75 wt % recovery of the non-ionic organic compound can be achieved. By integration of Recoflo Technology with the acid retardation resin, minimal dilution of the two product streams can be achieved.

The recovered acid could, be directly reused in the hydrolysis step. However, if re-concentration where to be needed, this could be done either by evaporation of water or by adding more concentrated acid.

### Anionic Exchange or Exclusion Chromatography

Acidic can be separated from sugars by using a bed of anionic exchange or exclusion chromatographic material. Due to the resin being of anionic material, it is the acid which will adsorb onto the solid phase. Therefore, a series of fractions containing the sugars which will elute first and a series of fractions with the acid will elute later, after elution with water.

This method of separation with an anionic resin and acid adsorption, results in the acid being obtained at a higher concentration and purity when compared to methods where cationic chromatographic material is utilized. This difference is important from an energy- and economic perspective, as the re-concentration of acid is significantly more expensive than the method for concentrating the sugar stream. Also, the anionic solid phase was employed as a simulated moving bed separation unit, which allows for a continuous separation system.

Due to the attained sugar stream being more diluted with this method than with a cation resin bed, there is need of concentrating the sugar solution before for example fermentation. This can be done by application of heat or vacuum. However, application of heat may be more expensive and lead to further degeneration of the sugars. The use of a filter or of a reverse osmosis membrane may be a more economical alternative, with the reverse osmosis option having a feasible operational concentration range of 15-16% and the sugar having a preferred concentration of 12-22%.

The separation is optimal at around 60° C., but can be employed in a span between room temperature to 80° C. After separation, the bed is washed with water and the acid fractions are combined, concentrated and recycled for reuse.

### Ion Exchange Chromatography (Cationic Resin)

Cation exchange chromatography is a well known method for separating acids from sugars. A strong acid resin heated to 40°-60° C. can be used, onto which the sugars become adsorbed. When the acid has eluted, with a flow rate of 2-5 mph., a gas with preferably less than 0.1 ppm dissolved oxygen, is blown into the resin bed to elute any remaining acid. The resin is then washed with water, preferably containing less than 0.5-0.1 ppm dissolved oxygen, to produce a sugar-rich stream. The sugar yield can be as high 98% of the sugar present in the hydrolysate. The produced sugar stream typically consists of 15% sugar and no more than 3% acid.

In contrast to ion exclusion chromatography, ion exchange chromatography does require regeneration of the resin as ion exchange does take place. The resins for this chromatography method are usually classified as strongly or weakly acidic/basic. The resin can for example be treated with sulfuric acid to produce a strongly acidic resin bed. One of the major economic weaknesses of conventional cation-exchange chromatography is related to the long cycle times necessary. The long cycle times entails an extended amount of time during which the resin is exposed to the acid, resulting in short resin lifespan. A possible way to tackle this disadvantage is to employ short cycle times and frequent resin washes Another drawback of the method is the presence of the divinylbenzene cross-links, which serve to stabilize the resin structure, as these may interact with the acid in an oxidative manner.

### Molecular Weight Cut Off by Ultrafiltration

In addition to finding a way to separate the acid from the glucose after hydrolysis, it is of interest to separate shorter cellulose chains from bigger ones. Such a method may be applied e.g. after the first step in the two-step acid hydrolysis of the present disclosure. This would help resolve the issue of degrading any cellulose chains too far during the second hydrolysis step and would be especially beneficial in the case of a cotton/viscose feedstock. Methods of molecular weight cut-off (MWCO) may thus be integrated in the process. Factors which must be taken into consideration with MWCO methods are e.g. the composition of the sample, molecular weight and shape, concentration of the sample and operation conditions such as cross-flow velocity, temperature and pressure. For efficient separation of two types of molecules with different molecular weights, it is recommended for the solutes to differ with a factor of ten between their molecular masses. Another rule of thumb for efficient separation is that the MWCO rating of the membrane must be a minimum of one-half of the solute for it to be retained. Ultrafiltration (UF) and nanofiltration (NF) are MWCO methods where UF is used for removal of macromolecular species such as polysaccharides, and NF is employed for removal of monosaccharides. UF is non-denaturing and considered more flexible and efficient than alternative methods. Some advantages of low-pressure UF are that it comprises a compact plant and process, that there is no need for chemicals and that there is a constant quality of the particle removal.

UF membranes retain particles ranging between 1,000-1,000,000 molecular weight. Viscose fibres usually have a DP between 200-300 and cotton usually have a DP between 3,000-4,000. If it is presumed that the initial acid-treatment only affects the viscose fibres and not the cotton fibres, the difference between the DP values of these solutes may range between the tenfold to the hundredfold, which would make ultrafiltration a plausible separation method for viscose and cotton fiber, depending on the molecular weights.

### Optimal pH for Catalytic Conversion of Glucose Solution

The sugar solution produced in the present disclosure can be converted to various organic chemicals by catalytic and biocatalytic processes. The optimal pH for a fermentation process varies dependent on desired end product, process and catalyst design. The pH in the steps following saccharification can be controlled by recycling more or less acid or by adding a neutralising agent (alkali or lime) to the sugar solution.

As one objective with the process disclosed herein is to enable recycling of used viscose, cotton and cold alkali fibers into new textile fibers the target molecules for fermentation are butanediol or caprolactam, which both are used widely to manufacture textile fibers such as spandex, lycra and nylon fibers.

Bio-butanediol is typically produced through fermentation of glucose by bacterial species such as Bacillus polymyxa, E. coli or Klebsiella pneumoniae.

Caprolactam have a number of alternative production routes. It may be derived from glucose, either by fermentation into the intermediate product Lysine, with the Corynebacterium glutamicum bacteria, or by conversion into the intermediate product levulinic acid. In either case, the pH of the glucose feedstock is of importance for the production rate. To obtain the optimal pH for product formation, the glucose purity generated by the acid-recovery process is an important aspect. An economical trade-off situation may arise between the cost of the acid-recovery method and the effectiveness of the glucose-to-end-product formation.

In the case of butanediol production the following operating data is typical; Fermentation with the Klebsiella sp. Zmd30 strain has an optimal pH of 6.0 and a yield of 82-94% (depending on the trade-off with the productivity) and fermentation with Klebsiella oxytoca NBRF4 has an optimal pH of 4.3 which entailed a yield of 0.32 g/g in one study, and an optimal at pH 6.3 with a yield of 0.37 g/g according to another study.

Production of caprolactam by fermentation is typically performed in a pH range of 7 to 8.

The acid hydrolysis is preferably performed in a two-step procedure wherein the waste textile material is treated with concentrated sulfuric acid in a first step, followed by treatment with diluted acid. The concentration of acid in the first step is from about 60 to 80% and in the second step from about 5 to 15%.

Heterogeneous solid acids, further discussed below, can partially or fully replace any homogeneous acid such as sulfuric acid in the hydrolysis step.

### Solid Acids for Catalytic Hydrolysis Step

Mineral acids, such as HCl and H₂SO₄, have been used in the hydrolysis of cellulose. However, they suffer from problems of product separation, reactor corrosion, poor catalyst recyclability and the need for treatment of waste effluent as allude to herein. The use of heterogeneous solid acids can solve some of these problems through the ease of product separation and good catalyst recyclability. Solid acids can with advantage be used to provide the acidity in the hydrolysis (saccharification) step of the present disclosure. The acid strength, acid site density, adsorption of the substance and micropores of the solid material are all key factors for effective hydrolysis processes. Methods used to promote reaction efficiency such as the pre-treatment of cellulose to reduce its crystallinity or microwave irradiation to improve the reaction rate can be applied to further enhance the catalysis.

### Metal Oxides

Several types of solid acids can be used in the practice of the present disclosure including metal oxides, it is for example known that mesoporous Nb—W oxide could be used as a solid catalyst for depolymerisation of cellulose (HNbMoO₆.) The high activity of HNbMoO₆ is attributed to its strong acidity, water-tolerance and intercalation ability.

In addition, nanoscale metal oxide catalysts have the potential to improve the catalytic performance of the hydrolysis reaction. In experiments Nano Zn—Ca—Fe oxide gave better performances with respect to hydrolysis rates and glucose yields than fine particle Zn—Ca—Fe. Besides, the paramagnetic nature of Fe oxides make it easy to separate the nano Zn—Ca—Fe oxide from the reaction mixture by simple magnetic filtration techniques.

### Polymer Acids

Polymer based acids with Brønsted acid sites are effective solid catalysts for many organic reactions including acid hydrolysis of cellulose. Apart from the well-known Amberlyst-type resins such as for example Amberlyst RTD, also Nafion (sulfonated tetrafluoroethylene based fluoropolymer-copolymer) are effective solid acid catalysts for the hydrolysis of cellulose in accordance with the present disclosure.

Sulfonated chloromethyl polystyrene resins sa CP—SO₃H containing cellulose-binding sites (—Cl) and catalytic sites (—SO₃H) are particularly effective in depolymerising cellulose structures Cellobiose could be completely hydrolyzed in 2-4 hours at 100-120° C. by CP—SO₃H, and microcrystalline cellulose (Avicel) could be hydrolyzed into glucose with a yield of 93% within 10 hours at moderate temperature (120° C.).

Low activation energy allows the CP—SO₃H-catalyzed hydrolysis to proceed at low temperature, which reduces energy consumption and avoids undesirable sugar degradation. The low activation energy of CP—SO₃H might be attributed to its ability to adsorb/attract cellobiose and cellulose and to disrupt hydrogen bonds of cellulose.

Solid acids of most interest for the hydrolysis of cellulose are those which are carbon-based and can be considered cellulose “mimetics”. This is due to their thermal stability, reusability, environmental friendliness, stronger catalytic activity and lower price. In particular, the polystyrene based sulfonated polymers are favourable. The solid acid resin CMP-SO₃H, often called the Pan catalyst is composed of a sulfonated chloromethyl styrenic-polymer (a CMP polymer) which is aromatic-rich with —Cl binding-sites and —SO₃H catalytic-sites can advantageously be used as solid acid in the hydrolysis step of the present disclosure. Cl— binding sites not only form very strong hydrogen bonds with the cellulose but also enhances the dissolution of the cellulose by disrupting its inter- and intra-hydrogen bonds.

### Sulfonated Carbonaceous Based Acids

Among various types of solid acid for the hydrolysis of cellulose, carbonaceous solid acids have superior catalytic activities. The good recyclability and cheap naturally occurring raw materials of these carbonaceous acids make them good candidates for commercial application. They can be manufactured by incomplete carbonization at low temperature to form small polycyclic aromatic carbon rings which are subsequently sulfonated with sulfuric acid to introduce sulfonic groups (—SO₃H).

### Heteropoly Acids

Heteropoly acids (HPAs) are a type of solid acid, consisting of early transition metal-oxygen anion clusters, and they are usually used as a recyclable acid in chemical transformations. The most common and widely used heteropoly acids are the Keggin type acids with the formula [XYxM₍₁₂₋ₓ₎O₄₀]ⁿ⁻ (X is the heteroatom and M and Y are addendum atoms).

Heteropoly acids have received much attention due to their fascinating architectures and excellent physicochemical properties such as Brønsted acidity, high proton mobility and good stability. They dissolve in polar solvents and release H, whose acidic strength is stronger than typical mineral acids such as sulfuric acid. However, the Keggin type acids cannot be used as heterogeneous catalysts in polar solvents. The substitution of protons with larger monovalent cations such as Cs⁺ gives solid catalysts that are insoluble in water and other polar solvents. This complicates the use of HPAs in conjunction with hydrolysis of cellulose.

### H-Form Zeolites

Zeolites are microporous, aluminosilicate minerals that are commonly used in petrochemistry. They are non-toxic and easy to recover from solution. They have porous structure that can accommodate a wide variety of cations, such as H+, Na+, K+, Mg2+. These cations are loosely bonded to the zeolite surface and can be released into solution to exhibit different catalytic activities. H-form zeolites are widely used acid catalysts due to their shape-selective properties in chemical reactions.

The acidity is related to the atomic ratio of Si/Al; the amount of Al atoms is proportional to the amount of Brønsted acid sites, the higher the ratio of Al/Si, the higher the acidity of the catalyst.

### Magnetic Solid Acids

For a practical process for the hydrolysis of cellulosic viscose, cotton and cold alkali fibers to glucose using solid acids as catalysts, a challenge may exist with respect to the catalyst recycle. Solid catalysts cannot be directly separated and recycled if there is a substantial solid fiber residue. To address this problem we are suggesting the use of magnetic solid acids sa magnetic sulfonated mesoporous silica (Fe₃O₄-SBA-SO₃H).

### Process Design

Glucose is the target molecule for the textile waste hydrolysis step. The glucose yield should preferably be higher than about 90%, preferably higher than 95% calculated on sugar content of raw material. As the only carbohydrate in waste textile material is cellulose, the operating parameters in saccharification can be tuned to optimize the yield of sugar monomers, primarily being glucose.

The target is to achieve as high glucose concentration as possible after hydrolysis step, and concentrations in the 30-50 g/l is feasible by controlling hydrolysis parameters. Reducing the dilution in the glucose production step is one method to achieve glucose concentrations above about 50 g/L, although this may come at the cost of reducing the acid hydrolysis glucose yield.

In one example, textile waste material comprising both cellulosic fibers and polyester is pre-treated hydrothermally prior to saccharification in an aqueous solution at elevated temperature, optionally in the presence of additives and an acidic catalyst. Any fibrous polyester material can be separated from cellulosic material for re-use prior to charging hydrothermally treated material to a fermentation step, or alternatively let the polyester pass through the processing steps as an inert.

Alternatively, or combined with hydrothermal treatment, the waste textiles may be pre-treated by a steam explosion procedure wherein the waste textile raw material is treated with hot steam, for example having a temperature of 180° C. to 240° C., and optional acidic catalysts under a pressure ranging from 1 to 3.5 MPa, followed by an explosive decompression to atmospheric pressure. This results in a rupture of the cellulosic textile material rigid structure, changing the starting material into a fibrous dispersed solid. Another pretreatment procedure may comprise treatment with supercritical CO₂, wherein the liquid CO₂ is used as a solvent for decomposed colorants.

The fashion industry has already started to show an interest in fiber recycling to reduce the environmental impact associated with waste textiles. Even though there are no commercial-scale recycling processes available, some small-scale projects have been initiated to recycle waste textiles into fibers that can be spun again. However, fibers cannot be recycled indefinitely because their properties (water absorption, tensile strength, etc.) degrade with each recycling loop, and therefore end-of-life valorization techniques will be needed for fibers that have already been recycled several times. Moreover, certain textiles already have a depolymerized cellulose chain (corresponding to an intrinsic viscosity IV lower than about 600 as determined by using ISO5351:2010) in the virgin garment such as viscose, carbamate, and other cold alkali fiber textiles and such textile fibers cannot efficiently be recycled or regenerated into new textile fibers. In this context organic chemicals production provides an attractive alternative to valorize such waste textile material.

Previous research on ethanol production from waste cellulosic cotton and viscose textiles has been focused mainly on developing pre-treatment procedures that can increase the amenability of the material to bioconversion, and therefore increase the biofuel yields from this waste. Cotton and viscose textiles are not amenable to direct saccharification and fermentation through biological means as: i) cellulose from cotton has a high crystallinity index; ii) dyes bond covalently with the surface of the cellulose, which reduces the accessible area for the enzymes; iii) and in mixed fiber textiles including both cellulosic fibers such as cotton and chemical fibers such as polyesters, polyesters reduce the accessibility of the enzymes to the cotton fibers.

Pretreatment using various solvents has been suggested as a successful strategy to deliver high bioethanol yields from waste cotton. For example, Jeihanipour et al. (Waste Management, 30, pp. 2504-2509, 2010, “A novel process for ethanol and biogas production from cellulose in blended-fibers waste textiles” showed that 80-85% overall bioethanol yields, based on the energy values of the raw material and the product, could be achieved through pretreating waste textiles with an organic solvent (NMMO). However, the economics of such technologies remain unclear because extremely high recoveries of the solvent (around 99%) would be required to render the process economically feasible.

To avoid the solvent-recovery loops, pretreatments with different chemicals have been explored as an alternative. Satisfactory results have been obtained for both pretreatment with acids and with bases, although an enzymatic hydrolysis step was always used prior to ethanol fermentation. For example, Shen et al. (Bioresource Technology, 130, pp. 258-255, 2013, “Enzymatic saccharification coupling with polyester recovery from cotton-based waste textiles by phosphoric acid pretreatment”) obtained an 80% overall bioethanol yield through pretreating with phosphoric acid”. Hasanzadeh et al. (Fuel, 218, pp. 41-48, 2018, “Enhancing energy production from waste textile by hydrolysis of synthetic parts”) used a pretreatment with sodium carbonate that delivered 60% overall bioethanol yield”.

Acid hydrolysis saccharification with sulfuric acid or sulfur dioxide in a two-step sequence or hydrolysis in the presence of a solid acidic are preferred procedures for depolymerization of the cellulosic polymers to glucose monomers in accordance with the present disclosure. If enzymatic saccharification is used, the enzymes are at least partially recycled in a preferred example.

Effluents from any of the process steps of the present invention such as pretreatment procedures, or saccharification and fermentation steps as described herein can, after optional neutralization, advantageously be charged to a kraft mill chemicals recovery cycle or to a pulp mill secondary effluent treatment plant.

Other methods of pretreating the raw textile material such as shredding, de-colorization and separation of buttons/zippers, separation of non-cellulosic material including polyester fabric etc. may also be performed prior to saccharification on or off site.

If deemed necessary, de-colorization or de-inking of textile material may be performed by standard procedures well known in the art.

Once the cellulosic material in the textile feedstock is depolymerized/saccharified, the sugar solution such as glucose solution can be transformed by catalytic, biocatalytic or microbial processes to valuable organic chemicals.

Manufacturing of ethanol by fermentation of sugars can be performed on the sugar solution of the present disclosure by any known procedure using yeasts such as Saccharomyces cerevisiae. While ethanol production is preferred as an intermediate step for obtaining hydrocarbon distillate fuels from cellulosic textile wastes in accordance with the process of the disclosure, also other alcohols such as butanol or isobutanol can be synthesized for subsequent upgrading to distillate fuels such as jet fuels by procedures well known to the artisan.

Following an acidic hydrolytic saccharification of the cellulosic textile fibers the sugar solution prepared becomes acidic. While the pH can be adjusted, the solution can directly or indirectly be transformed by microbial processes to chemical intermediates such as 1,4 butanediol, 2-propanediol, isobutanol, isoprene and caprolactam or to proposed platform chemicals such as FDCA (2,5 Furan dicarboxylic acid), succinic acid, glucaric acid 3-hydroxypropionate, lactic acid and malonic acid.

1,4-Butanediol (BDO) is an important commodity chemical used to manufacture over 2.5 million tons annually of plastics, polyesters and spandex fibers annually. BDO is currently substantially produced through intermediates derived from oil and natural gas such as acetylene, butane, propylene and butadiene. Given the importance of BDO as a chemical intermediate and issues associated with petroleum feedstocks, alternative low-cost manufacturing routes from sugars have been highly sought after.

Biocatalytic process are currently commercialized for the manufacturing of BDO from renewable carbohydrate feedstocks, based on biocatalysts such as engineered Escherichia coli capable of producing over 20 g/l of this highly reduced, non-natural chemical. E. coli microorganisms has recently been developed that allows for efficient anaerobic operation of the oxidative tricarboxylic acid cycle, thereby generating reducing power to drive the BDO pathway. Such engineered organisms can produce BDO from glucose solutions derived from waste cellulosic textile fibers.

2,5-Furan dicarboxylic acid (FDCA) is an organic chemical compound consisting of two carboxylic acid groups attached to a central furan ring. 2,5-Furan dicarboxylic acid (FDCA) can be produced from certain carbohydrates and as such is a renewable resource. Furan-2,5-dicarboxylic acid (FDCA) has been suggested as an important renewable building block because it can substitute for terephthalic acid (PTA) in the production of polyesters and other current polymers containing an aromatic moiety. FDCA has also large potential in the manufacturing of PEF (Polyethylene 2,5-furan-dicarboxylate), also named polyethylene furanoate and poly (ethylene furanoate). PEF is a polymer that can be produced by polycondensation of 2,5-furan-dicarboxylic acid (FDCA) and ethylene glycol.

PEF exhibits an intrinsically higher gas barrier for oxygen, carbon dioxide and water vapor than PET and can therefore be considered an interesting alternative for packaging applications such as bottles, films and food.

The versatility of FDCA is also seen in the number of derivatives available via relatively simple chemical transformations. Selective reduction can lead to partially hydrogenated products, such as 2,5-dihydroxymethylfuran, and fully hydrogenated materials, such as 2,5-bis(hydroxymethyl)tetrahydrofuran. Both these latter materials can serve as alcohol components in the production of new polyester, and their combination with FDCA would lead to a new family of completely biomass-derived products.

A key step in the manufacturing of FDCA from the sugar or glucose solutions manufactured from textile wastes in accordance with the present disclosure is a catalytic dehydration step. Other routes to FDCA via oxidation of hydroxymethylfurfural (HMF) with air over different catalysts have been explored.

Yet another example of specific and advantageous use for the sugar solution prepared in accordance with the disclosure is the manufacturing of caprolactam by microbial and/or fermentation processes. Caprolactam is a platform chemical that is used for production of Nylon 6, a fiber used in for example carpets and clothing with a current global market of more than 5 million t/y.

Other advantageous use of the sugar solution recovered from the hydrolysis step of the present disclosure is to produce lactic acid, succinic acid, or malonic acid. Lactic acid is primarily used for the manufacturing of PLA (polylactic acid) a bioplastic. It can also be converted to acrylic acid by a catalytic process. Acrylic acid is mainly used for production of polyacrylate fibers.

Succinic acid may be used for production of PBS (polybutylene succinate) that in turn may be used in textile applications. Malonic acid can be manufactured from the sugar solution for example by fermentation with a modified yeast (polyketide synthases). Malonic acid and its derivatives malonates can be used in coating applications.

### Examples Relating to Jet Fuel Production

Even though ethanol may be used directly as an energy carrier, one of the objectives of this disclosure is to provide distillate fuels such as jet fuels sourced from the waste textile material. To transform the alcohol to distillate fuels, the alcohol is first dehydrated over a catalyst. Suitable catalysts include zeolites, SAPO catalysts, activated clay, phosphoric acid, sulfuric acid, activated alumina, transition metal oxides, transition metal composite oxides, and heteropolyacid catalysts.

To remove the water present in the dehydration reactor, the effluent stream may be condensed by cooling the entering gas with spray water. This allows the separation of the olefin from the undesired products, including water, impurities, and unconverted alcohol. At this stage, the olefin contains small amounts of CO₂ that needs to be removed before drying the olefin and thus obtain a gas that does not contain water. Once this step is conducted, the remaining impurities can be removed, for example in a cryogenic distillation column.

In the case of ethanol being the primary feedstock, ethylene will be formed which subsequently is transformed and oligomerized in a second catalytic process to linear alpha-olefins. Producing isobutene directly from ethanol as an intermediate olefin prior to oligomerization to fuel-range hydrocarbons represents an alternative to the ethylene route. The advantages of using isobutene as an intermediate in this way include easy conversion of isobutene into its dimer, diisobutene, which is a highly branched high-octane product that can be blended into gasoline, and more selective conversion of isobutene to a specific targeted hydrocarbon range. Ethylene oligomerization when using zeolites typically requires activation by strong Brønsted acid sites at higher reaction temperatures, thus making selectivity control difficult to perform in one step.

ZnₓZr_{y}O_{z} mixed-oxide type catalysts with balanced acid-base sites can advantageously be used for converting ethanol to isobutene in a one-step process.

Once the olefins which are the building blocks for the production of jet fuel are formed through dehydration of alcohols, these intermediates are further converted at moderate temperatures and pressures, for example at a temperature of 150-250° C. and a pressure of 3-4 MPa, into a middle distillate that contains diesel and kerosene via oligomerization.

Distillate, ready-to-use fuel in the jet range is made from these oligomers by hydro-treating and isomerization to branched alkanes. The middle distillates produced through these processes may as a final step undergo distillation to obtain the range of paraffins and other compounds that meet the standard fuel specifications for aviation purposes. The latter synthesis steps are preferably performed in a petroleum refinery environment.

The jet fuel range is defined herein by the carbon numbers of the hydrocarbons which shall be in the range of C8-C16.

### FIGURES

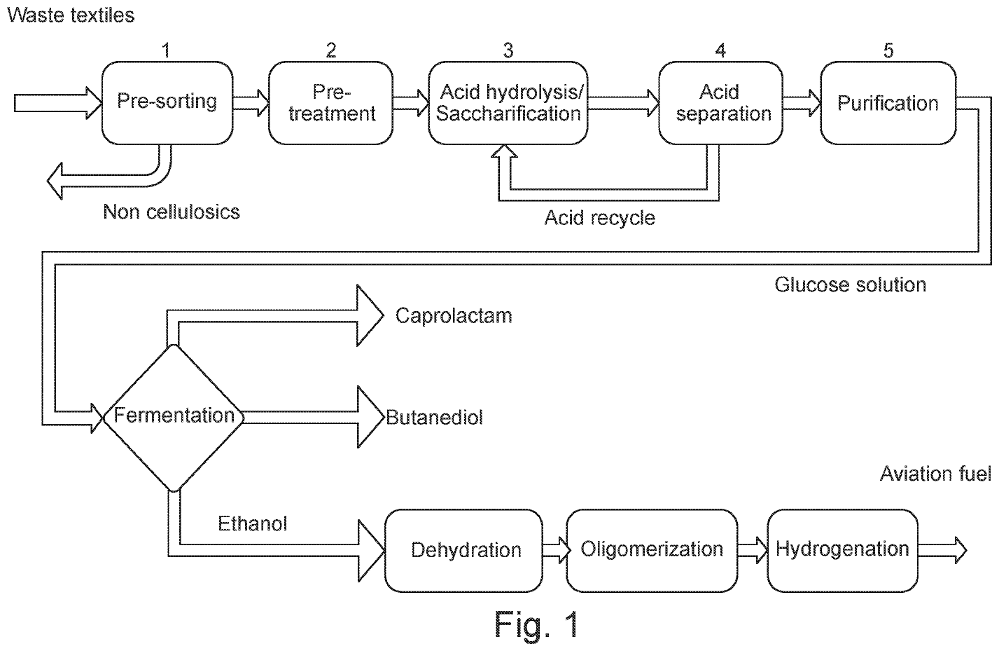

A process for manufacturing organic chemicals and/or distillate hydrocarbon fuels from waste textiles comprising cellulosic fibers according to an example is described with reference to FIG. 1.

In step 1 in FIG. 1, pre-sorting of mixed textile waste material by means of for example of visual (VIS) and near-infrared (NIR) spectroscopy is performed. Different types of textile fibers, such as cotton, wool, viscose, polyester and acrylic can be identified and separated into distinct streams. This unit operation can be performed at any distant location or integrated with the other process steps of the present disclosure. The output from pre-sorting of specific interest for the process are all cellulosic fibers, such as cotton, viscose, cold alkali fibers etc., and more specifically worn out cotton, viscose and cold alkali fiber fabrics, wherein the cellulosic polymer chains have an average intrinsic viscosity below about 600 as determined by ISO5351:2010.

In step 2 in FIG. 1, a cellulosic textile waste stream may optionally be pre-treated by, for example, grinding, chopping, cutting, steam explosion treatment or hydrothermal treatment prior to further processing. This optional step opens the fabrics and increase the accessibility of hydrolysis catalyst in the following acid hydrolysis step. The objective with this step is to form a slurry of fabric particles and fiber wherein the particles are smaller than about 10×10 mm in area.

In step 3 in FIG. 1, the cellulosic textile waste stream is charged into a two-step acid hydrolysis reactor system wherein the glycosidic bonds of the cellulosic polymers are broken and glucose as a monomer sugar is formed. The first step is performed with high acid concentration and the second step with lower acid concentration to minimize formation of undesired decomposition products and to increase the yield of glucose. The acid catalyst is preferably sulfuric acid. The homogeneous acid can be partially or fully replaced by a heterogeneous acidic solid catalyst such as for example Amberlyst 15. A slurry of spent catalyst, glucose and decomposition by-products are formed.

In step 4 in FIG. 1, the slurry from the acid hydrolysis step is charged to a separation unit that may be directly integrated with the hydrolysis step. In the separation unit, a substantial fraction of the homogeneous acid catalyst, and/or the heterogeneous solid acid catalyst, is separated from the glucose rich sugar solution. The catalyst is purified and restored if needed and is together with makeup catalyst recycled to the acid hydrolysis step

In step 5 in FIG. 1, the glucose solution obtained is further treated, adjusted for correct pH concentrated and purified if needed to a level necessary for downstream use as feedstock for manufacturing of organic chemicals and/or distillate hydrocarbons.

In a following step in FIG. 1, glucose solution is fermented in the presence of biocatalysts in accordance with well know procedures to yield, for example, ethanol, bio 1-4 butanediol or bio-caprolactam. The products are further purified, concentrated for conversion to for example spandex fibers, in the case of butanediol, or nylon 6 in the case of caprolactam.

Also, other fine organic chemicals such as succinic acid, lactic acid and malonic acid can directly or indirectly be produced from the glucose rich sugar solution recovered from step 4

Alternatively, ethanol, a classical product from fermentation of glucose, is produced. Ethanol have many uses, and one recent application of ethanol is for the manufacturing of aviation fuel over dehydration, oligomerization, and hydrogenation. Other uses of dehydrated ethanol include the manufacturing of ethylene and polyethylene.

The use of saccharification enzymes for hydrolysis of cellulose into glucose is well known art and not further discussed here.

It is contemplated that there are numerous modifications of the examples described herein, which are still within the scope of the disclosure as defined by the appended claims.

### Examples Section

Below, certain examples of the present disclosure are presented and summarized.

In a first example the present disclosure is directed to a process for the manufacturing of fine chemicals and/or distillate hydrocarbon fuels in the jet fuel range from textile waste comprising cellulosic fibers, wherein the process comprises the following steps:
providing a stream of waste textiles or pre-processed waste textiles.
disintegrating waste textiles by a chemical, thermochemical or mechanical treatment into an aqueous slurry of comminuted waste textiles.
saccharification of the cellulosic polymers in the waste textiles by acid hydrolysis or by treatment with saccharification enzymes into monomer sugars; and
fermentation or catalytic conversion of the monomer sugars to an alcohol and/or to organic fine chemicals.

According to one example, the process is directed to the manufacturing of distillate hydrocarbon fuels in the jet fuel range, and wherein the process comprises:
fermentation of the monomer sugars forming an alcohol.
separating the alcohol and concentrating the alcohol by distillation; and
further treating the concentrated alcohol in one or more steps to form distillate hydrocarbon fuels in a carbon number range of C8 to C16.

According to another example, the process is directed to the manufacturing of organic fine chemicals, and wherein the process comprises:
fermentation and/or catalytic conversion of the monomer sugars to fine chemicals, e.g. 1,4 butanediol, caprolactam, succinic acid, lactic acid, and malonic acid.

Moreover, according to yet another example, the process comprises biocatalytic conversion of monomer sugar solution monomer sugar solution to 2,5 furan dicarboxylic acid.

Furthermore, according to another example, pre-processing of waste textiles comprises mechanical and/or chemical separation of polyester and/or cotton textiles from the stream of waste textiles, preferably prior to saccharification.

According to one example of the fermentation route, the alcohol is ethanol.

Moreover, the step of further treating the concentrated alcohol may comprise at least one of dehydration, oligomerization and hydrogenation.

Furthermore, the step of further treating the concentrated alcohol may be performed in a petroleum refinery.

According to yet another example of the present disclosure, isobutene is produced directly from ethanol as an intermediate olefin prior to oligomerization.

According to one specific example, saccharification is performed by acid hydrolysis.

According to another specific example saccharification is performed with a homogeneous acid catalyst by acid hydrolysis in two steps, at different acid concentrations in each step, a first step at high acid concentration and a second step with low acid concentration, wherein the acid concentration in a first step is from 60-80% (dissolving step) and in a second step from about 5-15%.

According to another specific example saccharification is performed in the presence of a solid acid catalyst, which preferably after optional restoring and reactivation is recycled to the saccharification step

Furthermore, according to yet another example, the process is integrated in a kraft, sulphite or organosolv pulp mill.

According to one further example, there is also performed a pretreatment of the waste textile stream which is a steam explosion process.

Moreover, according to another example, there is also performed a pretreatment being a hydrothermal treatment.

Moreover, according to yet another specific example of the present disclosure, the waste textiles comprise cotton, viscose, lyocell and/or other cellulosic fibers.

In one example, cotton fibers are separated from the waste textile stream prior to charging the waste textile stream to the processes of the present disclosure, such cotton fibers can advantageously be converted to dissolving cellulose pulp and be further processed in to regenerated cellulosic fibers such as viscose fiber.

Furthermore, according to a further example of the present disclosure, the waste textiles are pretreated off-site in order to facilitate processing into an alcohol or fine organic chemicals on site.

The process according to at least on example of the present disclosure may also be used for textile wastes comprising polyester.

In this context, according to an example of the present process, the waste textiles comprise polyester or other chemical fibers, which polyesters or other chemical fibers form an inert sludge during the fermentation or biocatalytic conversion, which inert sludge is separated and further treated by chemical or thermal processes to recover an energy or material value.

Furthermore, according to one example, the obtained inert sludge comprising polyester or other chemical fibers is directly or indirectly pyrolyzed forming a gas, which is condensed to a hydrocarbon liquid.

According to another specific example, the hydrocarbon liquid is sent/transported to a petroleum refinery for hydroprocessing into distillate fuels.

Furthermore, according to yet another specific example, the inert sludge comprising polyester or other chemical fibers is used as a feedstock for preparation of new chemical fibers.

### Acid Hydrolysis of Cotton, Example of Procedures

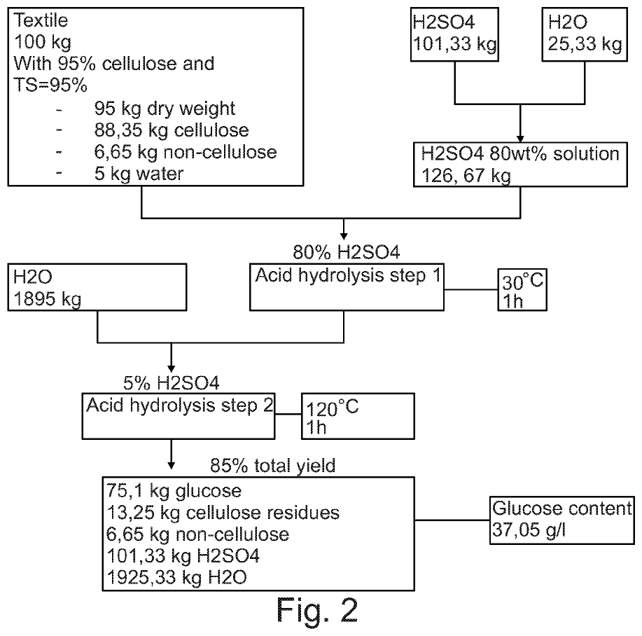

With reference to FIG. 2, a two-step hydrolysis process with weight contents of each step of the process will now be described in more detail. A two-step acid hydrolysis was performed on 100% cotton fabric with the objective of converting the cellulose-rich cotton into glucose. Further treatment of the obtained glucose may then be coupled to the process to produce green organic fine chemicals. With this procedure, textile waste may be recycled and used for a more sustainable production of ethanol, butanediol, caprolactam, or other value-added chemicals.

In the first step of the acid hydrolysis, the cotton samples were treated with 72% sulfuric acid at 30° C. for one hour during which they were stirred every ten minutes. A large portion of the cotton was dissolved. In the second step, the samples were diluted with water to achieve 5% sulfuric acid, after which the samples were heated to 120° C. for 1 h (2 h program with 1 h to cool).

Since the cellulose in the samples can become degraded further than to glucose, the so-called “glucose losses” had to be calculated. For this purpose, three samples of pure glucose were prepared, referred to as “sugar recovery standards”. Two of these samples were treated with the second step in the two-step acid hydrolysis and one glucose standard was saved without having undergone any acid hydrolysis. The reason for the glucose samples not having to undergo the first step of the acid hydrolysis is that the purpose of this step is to break down the cellulose into shorter chains, which is not needed for the glucose samples as they already constitute the shortest cellulose units.

After both steps of the acid hydrolysis, textile samples were filtered with vacuum filtration through a 20 μm porcelain filter. The filters with undissolved fibres were weighed before filtration as well as after having been both filtered and dried 12 hours at 105° C. From these weights, the mass of the undissolved material can be calculated. 2 ml of liquid from all nine samples (3 unwashed textile samples, 3 washed textile samples and 3 glucose samples) were filtered with 0.2 μm spray filtration and stored in eppendorf tubes in the refrigerator for HPLC analysis (High-performance liquid chromatography).

All samples were prepared for and run with HPLC with a hydrogen column to measure glucose content and possible degradation substances.

The total glucose yield of the process was obtained through the following calculations.

“Oven dry weight” (ODW) could be obtained from the weight (g). The solid residue (%) was then based on the ODW and describes the weight of non-cellulosic materials. The cellulose content was calculated using the three glucose standards, where two samples had undergone the second step of the hydrolysis process and one sample had been used as a reference point. “Sugar recovery” had an average value of approximately 95%. The glucose content was calculated through multiplying the Glucose dionex with the dilution factor (in this case the dilution factor=1) and dividing the product by the “sugar recovery”. Since the glucose content must be corrected with the amount of water in order to obtain the cellulose content, this quota was multiplied by 0.9. The starting cellulose content was divided by the corrected glucose content, multiplied by the volume and divided by the ODW, which gave “Glucan” (%), i.e. the percentage of the dry weight in the sample from start which consists of cellulose.

The sum of the weight of the liquid and the mass before the pre-treatment was related to the mass after the treatment, for calculation of “mass losses” (%), which amounted to approximately 3.7%. The weight of the cellulose after the pre-treatment was calculated as the mass of the solid multiplied by the dry matter content multiplied by the cellulose content. “Glucan losses” could then be calculated as:

1−the weight of the cellulose after the pre-treatment/cellulose content from the beginning (%)*weight of the textile used

This amounted to approximately 22%. Since 22%>>1.3%+3.7%, it can be concluded that this loss is due to more than just a human factor, that is, some of the textile had already been dissolved during the pre-treatment (glucose losses) and discarded with the wastewater. The total yield of the process, without any optimization procedures, is approximately 75%. That is, for a textile feedstock of 100 kg, 75 kg of glucose would be available for extraction in a 35.05 g/L concentrated solution after the two-step acid hydrolysis, as is shown in FIG. 2.

Combining the effects of concentrated and diluted sulfuric acid in a two-step hydrolysis results in a significant increase in glucose yield compared to one step hydrolysis. With optimization of the parameters of the two step procedure, a glucose yield of up to 92% of the theoretical maximum was achieved with a relatively low glucose concentration. The final glucose concentration was increased significantly through increasing the solids loading. At higher solids loading, the glucose concentration was approximately 40 g/L while the glucose yield was 84%. This increase in glucose concentration was achieved without any significant formation of byproducts. The glucose concentration was further increased to 50 g/L through other modifications of the procedure.

While the example procedure described above is shown for cotton as an example of a cellulosic fiber, initial experimental work has shown that other cellulosic fibers such as viscose and or cold alkali fiber fabric are easier to convert to glucose by acid hydrolysis. As expected, less severe conditions in terms of temperature, time and catalyst charge can be may be applied. Solid acids are also explored for partial or full replacement of the sulfuric acid with good initial results and much simpler catalyst recycle.

Experiments with saccharification enzymes gave a somewhat lower yield than two step hydrolysis (around 65%) and it was difficult to separate the enzymes from the hydrolysis broth.

1. A process for manufacturing organic chemicals and/or distillate hydrocarbon fuels from waste textiles comprising cellulosic fibers, wherein the process comprises:
providing waste textiles comprising cellulosic fibers;
processing the waste textiles into an aqueous slurry of comminuted waste textiles;
saccharification of the comminuted waste textiles into monomer sugars in the presence of a catalyst; and
processing the monomer sugars into organic chemicals and/or distillate hydrocarbon fuels.

2. The process according to claim 1, wherein saccharification of the comminuted waste textiles is performed by acid hydrolysis.

3. The process according to claim 2, wherein at least a portion of the acid hydrolysis is performed in the presence of a homogeneous and/or heterogeneous acid catalyst and wherein at least a portion of the acid catalyst after optional restoring is recycled to the saccharification step.

4. The process according to claim 1, wherein saccharification of the comminuted waste textiles is performed by treatment with saccharification enzymes.

5. The process according to claim 1, wherein processing of the monomer sugars to organic chemicals is performed by fermentation and/or catalytic conversion in the presence of a catalyst.

6. The process according to claim 5, wherein fermentation of the monomer sugars comprises fermentation of the monomer sugars to an organic alcohol, organic acid or to a lactam.

7. The process according to claim 6, wherein the process is directed to the manufacturing of distillate hydrocarbon fuels, and further comprises:
separating alcohol from a fermentation broth,
concentrating the alcohol by distillation; and
further treating the concentrated alcohol in one or more steps to form distillate hydrocarbon fuels in a carbon number range of C8 to C16.

8. The process according to claim 7, wherein the step of further treating the concentrated alcohol comprises at least one of dehydration, oligomerization and hydrogenation.

9. The process according to claim 6, wherein the alcohol is ethanol.

10. The process according to claim 9, wherein isobutene is produced directly from the ethanol as an intermediate olefin prior to oligomerization.

11. The process according to claim 1, wherein processing of the monomer sugars to organic chemicals is performed by fermentation or a catalytic conversion in the presence of a catalyst.

12. The process according to claim 11, wherein the monomer sugars are converted by fermentation and/or catalytic conversion in the presence of a catalyst to 1,4 butanediol, caprolactam, succinic acid, lactic acid, malonic acid or 2,5 furan dicarboxylic acid.

13. The process according to claim 1, wherein processing the waste textiles into an aqueous slurry of comminuted waste textiles is performed by a chemical, thermochemical, or mechanical treatment.

14. The process according to claim 1, wherein processing the waste textiles into a slurry of comminuted waste textiles comprises processing the waste textiles by at least one of chopping, milling, grinding and/or a steam explosion or a hydrothermal procedure.

15. The process according to claim 1, wherein the waste textiles are recovered from a pre-processing plant wherein the waste textiles a pre-processed by at least one of a steam explosion process, a hydrothermal treatment or a plant wherein recycled textiles are sorted into at least synthetic fabrics and cellulosic fabrics.

16. The process according to claim 15, wherein pre-processing of the waste textiles comprises mechanical and/or chemical separation of polyester and/or cotton fabric or fibers from the waste textiles.

17. The process according to claim 15, wherein the pre-processing of the waste textiles comprises mechanical sorting by fiber composition using by NIR/VIS technology (near infrared, visible ray).

18. The process according to claim 1, wherein the process is integrated in a kraft, sulphite or organosolv pulp mill.

19. The process according to claim 1, wherein the waste textiles comprise cotton (preferably low-quality cotton), viscose, lyocell cellulosic fibres, and/or cold alkali fiber or a mixture thereof.

20. The process according to claim 1, wherein the waste textiles charged to the process comprise a large fraction, preferably over 50% by weight of waste textiles, of cotton, viscose or cold alkali fibers having an average cellulosic polymer molecular chain length lower than corresponding to an intrinsic viscosity (IV) of 600.

21. The process according to claim 1, wherein the waste textiles further comprise synthetic fibers, and wherein during fermentation or catalytic conversion, the synthetic fibers form an inert sludge, the inert sludge being separated from the monomer sugars.

22. The process according to claim 21, wherein the synthetic fibers comprise polyester.

23. The process according to claim 21, further comprising pyrolyzing the inert sludge to form a synthesis gas, and condensing the gas to form a hydrocarbon liquid.

24. The process according to claim 1, wherein distillate hydrocarbon fuels have a carbon number of the hydrocarbons in the range of C8-C16.

25. The process according to claim 1, wherein the monomer sugars consist substantially of glucose.

26. The process according to claim 25, wherein a glucose yield of hydrolysis is higher than about 90% calculated on sugar content of raw waste textiles material.

27. The process according to claim 2, wherein the acid hydrolysis is performed with a homogeneous acid catalyst in two steps at different acid concentrations in each step, wherein the acid concentration in the first dissolving step is about 60-80% and in the second step is about 5-15%.

28. The process according to claim 2, wherein at least a portion of the acid hydrolysis is performed in the presence of a solid acid catalyst.

29. The process according to claim 28, wherein the solid acid catalyst is carbon based, in particular polymeric carbon body catalysts such as sulfonated polystyrene, for example CMP-SO₃H.
